(19) United States
(12) Patent Application Publication
(10) Pub. No.: US 2012/0141433 A1
(43) Pub. Date: Jun. 7, 2012

(54) Vaporized Stem Cell Derivatives for Topical and Other Therapeutic Uses

(76) Inventors: Nikolai Tankovich, San Diego, CA (US); Yuri Kudinov, San Diego, CA (US)

(21) Appl. No.: 13/251,781

(22) Filed: Oct. 3, 2011

Related U.S. Application Data
(60) Provisional application No. 61/390,592, filed on Oct. 6, 2010.

(51) Int. Cl. A61K 35/12; A61K 38/18; A61P 17/00; A61K 35/28

(52) U.S. Cl. 424/93.7; 424/577; 424/520; 514/8.1

(57) ABSTRACT

The invention provides compositions of vaporized stem cell derivatives and methods for their use and manufacture in the treatment of skin conditions and other therapeutic applications. Stem cell derivatives comprising vaporized stem cells, stem cell factors and/or stem cell microvesicles are disclosed and contemplated as being within the scope of the invention. The invention finds use in medical, rejuvenative and cosmetic applications.

### CROSS-REFERENCE TO RELATED APPLICATIONS

This application claims priority to provisional application Ser. No. 61/390,592 filed on Oct. 6, 2011, the entire contents of which are incorporated herein by reference.

### FIELD OF THE INVENTION

The present invention is in the field of stem cell therapy. In particular, the invention relates to methods of compositions employing preserved therapeutic microvesicles derived from stem cells. The invention further relates to methods of making the microvesicles, and methods for their use in a variety of therapeutic applications.

### BACKGROUND

Skin is subject to insults by many extrinsic and intrinsic factors. Extrinsic factors that can adversely affect the skin include wounds, ultraviolet radiation (e.g., from sun exposure), environmental pollution, wind, heat, low humidity, harsh surfactants, abrasives, and the like. Intrinsic factors that lead to skin problems include chronological aging, biochemical changes from within the skin, disease and medical disorders. Whether extrinsic or intrinsic, these factors result in visible signs of skin aging and damage, such as wrinkling, roughness and histological changes. As a result, the improvement of the appearance and health of the skin has become a concern for many people.

Extrinsic or intrinsic factors may result in the thinning and general degradation of the skin. For example, as the skin naturally ages, there is a reduction in the cells and the blood vessels that supply the skin. There is also a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990, which is incorporated by reference herein in its entirety. Moreover, the epidermis has a cell renewal system in which fully differentiated cells are continually shed from the skin surface and replaced by new cells formed within the germative cell layers of the skin. With increasing age, cell renewal rates decrease, leading to the development of coarse, sallow skin.

Skin also contains an elaborate network of elastin fibers that is responsible for maintaining its elastic properties. With excessive exposure to sunlight the elastic fiber system becomes hyperplastic, disorganized and ultimately disrupted. This process is known as actinic elastosis and it is a principal cause of wrinkling, discoloration and laxity of the skin in the exposed areas of the body. As new fibroblasts, endothelial cells and keratinocytes form, the skin can repair itself. However, the skin becomes less able to do so as it ages. Therefore, agents that can accelerate the growth and repair of prematurely aged skin are needed.

Methods and compositions for cosmetic treatment of the skin range from cosmetic creams and moisturizers to cosmetic surgery. However, while certain skin care compositions are available on the market, such compositions do not effectively stimulate the healing, growth, rejuvenation and overall health of new skin tissues. Hence, there is a need for a skin therapy that addresses these failures.

### SUMMARY OF THE INVENTION

An objective of the invention is to provide compositions comprising preserved stem cell derivatives for the treatment of skin conditions, and other therapeutic uses, wherein the preserved stem cell derivatives achieve enhanced storage and longevity properties over contemporary methods of preserving biologically active cell-based products.

### BRIEF DESCRIPTION OF THE DRAWINGS

FIG. 1 is a chart showing the results of foam ding on the stability of VEGF.

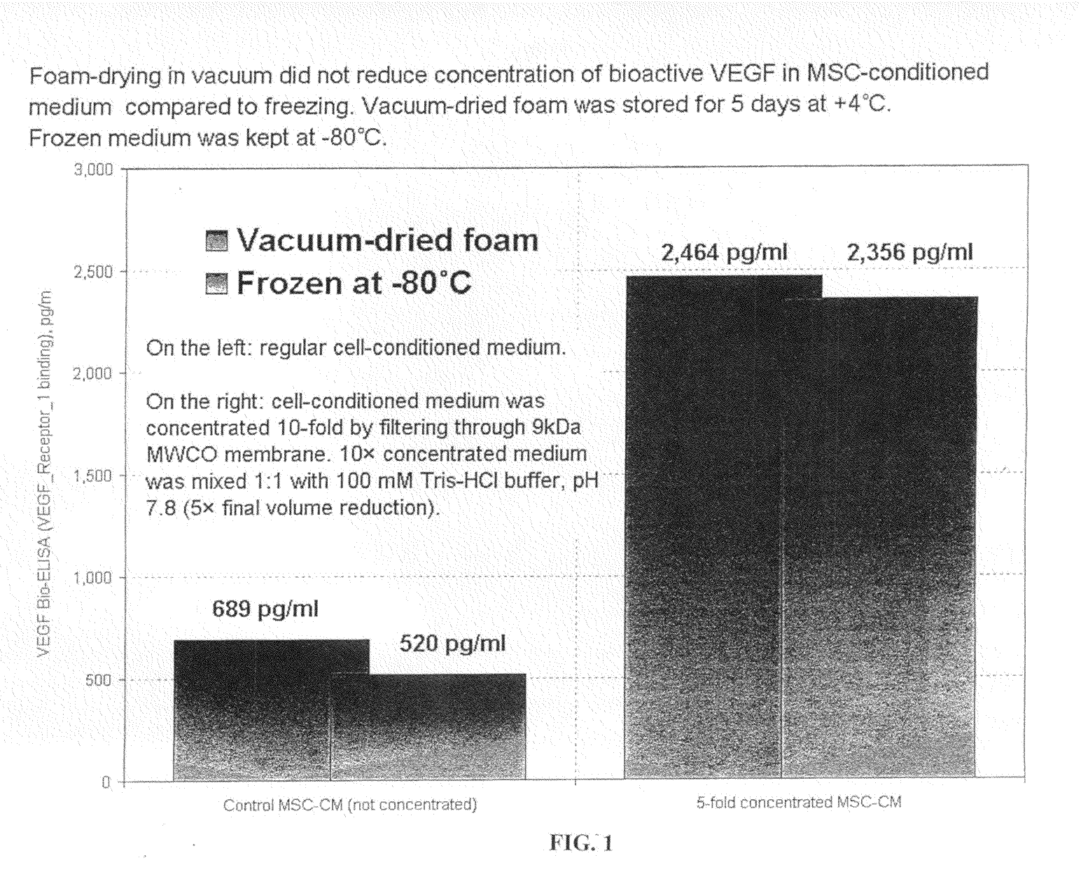

FIG. 2 is a chart showing the effects of the preservation solution on VEGF stability.

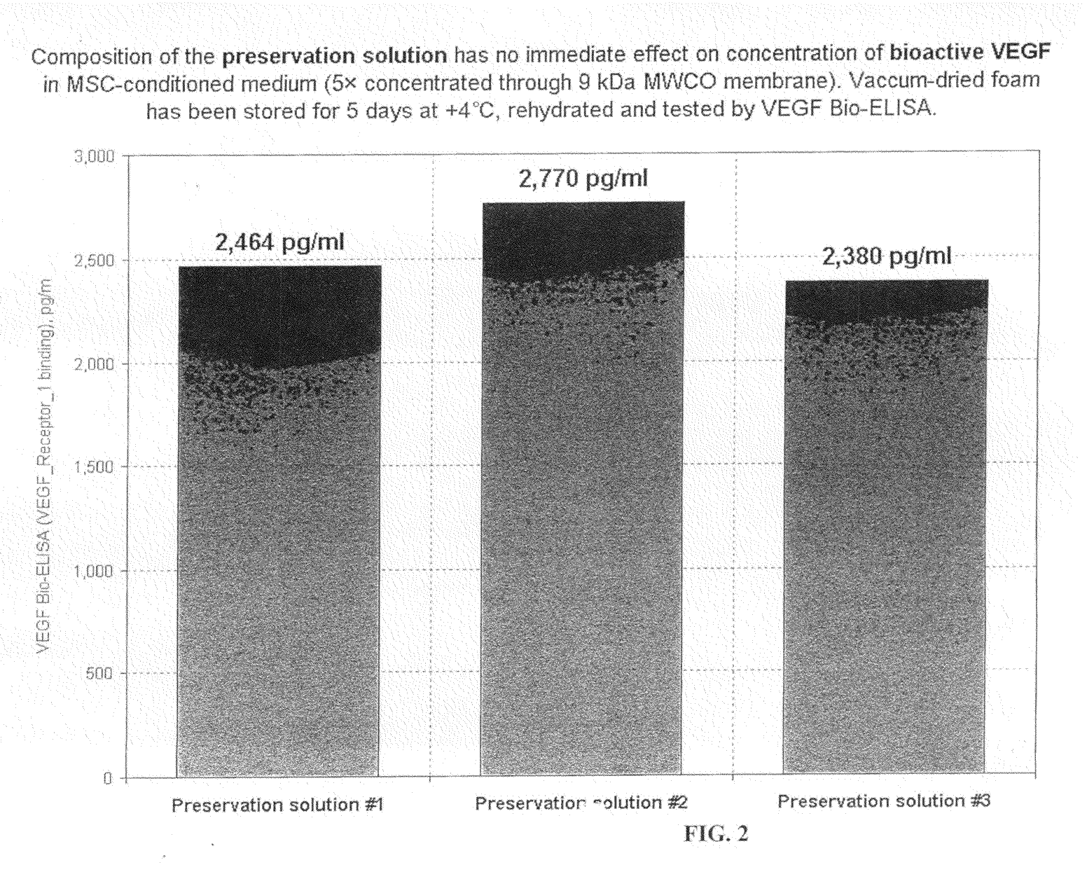

FIG. 3 is a chart showing the effect of foam-drying on the stability of VEGF.

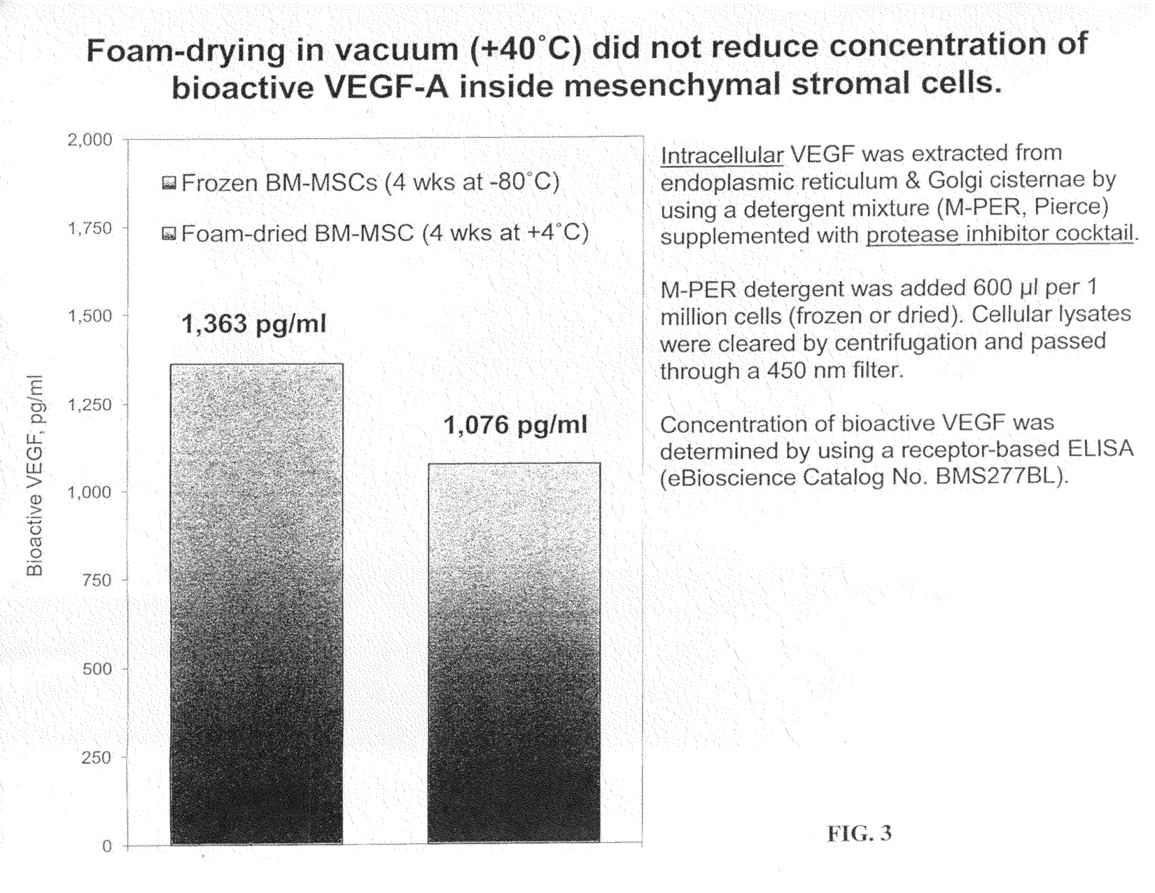

### DEFINITIONS

As used herein, the term “stem cell” refers to an undifferentiated cell which has the ability to both self-renew (through mitotic cell division) and undergo differentiation to form a more specialized cell, Stem cells have varying degrees of potency. A precursor cell is but one example of a stem cell.

The term “mesenchymal cell” refers to mesodermal germ lineage cells which may or may not be differentiated. The mesenchymal cells of the invention include cells at all stages of differentiation beginning with multipotent mesenchymal stem cells, down to fullly differentiated terminal cells.

The term “patient,” or “subject,” refers to animals, including mammals, preferably humans, who are treated with the pharmaceutical compositions or in accordance with the methods described herein

The term “pharmaceutically acceptable carrier” (or medium), which may be used interchangeably with the term “biologically compatible carrier” (or medium), refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, a “therapeutically effective amount,” or “effective amount,” refers to the number of transplanted cells which are required to produce a therapeutic effect or improvement in the disorder (e.g. skin condition) that is being treated.

The term “stem cell composition” refers to a composition containing whole stern cells, stem cell factors, microvesicles, or a combination thereof.

The term “stem cell derivative” includes, but is not limited to, biomolecules, cellular vesicles (e.g. secretory vesicles), whole stem cells (live or dead), cell organelles, membranes, and combinations thereof.

The term “biomolecule” refers to any molecule produced by a living cell including, but not limited to, proteins, peptides, amino acids, fatty acids and fatty acid alcohols, sterols, nucleic acids (e.g. DNA and/or RNA) and combinations thereof.

“Rejuvenating composition” refers to a composition that improves the appearance of, or rectifies, a condition (e.g. skin condition).

As used herein, “treating” or “treat” includes (i) preventing a pathologic condition (e.g. skin condition) from occurring; (ii) inhibiting the pathologic condition (e.g. skin condition) or arresting its development; (iii) relieving the pathologic condition (e.g. skin condition), or (iv) alleviating the symptoms associated with the condition (e.g. skin condition).

The term “clone,” or “clonal cell,” refers to a single cell which is expanded to produce an isolated population of phenotypically similar cells (i.e. a “clonal cell population”).

The term “cell line” refers to one or more generations of cells which are derived from a clonal cell.

The terms “administration” and “administering” as used herein refer to the delivery of a stem cell composition by an administration route including, but not limited to, intravenous, intra-arterial, intramuscular, intraperitoneal, subcutaneous, intramuscular, topically, or combinations thereof.

### DETAILED DESCRIPTION

The invention relates to compositions comprising vaporized stem cell derivatives for topical use (e.g. the treatment of skin conditions), as well as other therapeutic uses. The invention further contemplates methods for making the compositions of the invention.

Stem cells for producing stem cell derivatives for use with the invention include mesnechymal stem cells (MSC). Such MSC may be obtained from prenatal sources, postnatal sources, and combinations thereof. Tissues for deriving a suitable MSC include, but are not limited to, bone marrow, blood (peripheral blood), dermis (e.g. dermal papillae), periosteum, synovium, peripheral blood, skin, hair root, muscle, uterine endometrium, adipose, placenta, menstrual discharge, chorionic villus, amniotic fluid and umbilical cord blood. Mesechymal stem cells may be derived from these sources individually, or the sources may be combined (before or after enrichment) to produce a mixed population of mesenchymal stem cells from different tissue sources.

Mesenchymal stem cell compositions for use with the invention may comprise purified or non-purified mesenchymal stem cells. Mesenchymal stem cells for use with the invention, and their methods of manufacture, are disclosed in the following references, the disclosures of which are incorporated herein by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113: U.S. Pat. No. 6,387,367; U.S. Pat. No. 7,060,494; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

In some aspects of the invention, stem cells (e.g. MSC) for providing stem cell derivatives for use with the invention are grown (i.e. cultured) under low oxygen conditions. Without being limited to any particular theory, culturing the stem cells under low oxygen conditions increases stem cell proliferation and enhances the production of stem cell factors beneficial in the regeneration (and rejuvenation) of tissues in vivo.

The term “low oxygen” as used herein refers to the cell culture conditions in which stem cells are grown in an environment (i.e. incubator) having reduced oxygen tension (i.e. any oxygen concentration that is less than atmospheric oxygen). Thus, the stem cells for use with the invention may be grown in an oxygen concentration that is below about 20%, preferably below about 15%, more preferably below about 5-10%, at sea level. Low oxygen conditions may be kept as close as possible to the normal physiological oxygen conditions in which a particular stem cell would be found in vivo.

In one embodiment, the low oxygen conditions comprise an ambient (e.g. incubator) oxygen condition of between about 0.25% to about 18% oxygen. In another embodiment, the ambient oxygen conditions comprise between about 0.5% to about 155% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures

Methods for manufacturing stem cells under low oxygen conditions as disclosed herein are available in the art, including the methods disclosed in the following publications, the disclosures of which are incorporated herein by reference. U.S. Pat. No. 6,759,242; U.S. Pat. No. 6,846,641; U.S. Pat. No. 6,610,540; J. Cereb. Blood Flow Metab. 2008 Sep. 28(9):1530-42; Stem Cells. 2008 May 26(5):1325-36; Exp Neurol. 2008 April 210(2):656-70; Mol. Cell. Neurosci. (2007), doi: 10.1016/j.mcn.2007.04.003; Experimental Neurology 170, 317-325 (2001); and Neurosignals 2006-07, 15:259-265. Although these references disclose particular procedures and reagents, any low oxygen culture condition capable of expanding stem cells according to the invention may be used.

In some aspects, the stem cell derivative at least one stem cell factor. As used herein, the term “stem cell factor” refers to cellular molecules which influence the growth, proliferation, commitment, and differentiation, for example, of other cells (e.g. stem cells) either in vivo or in vitro. Stem cell factors include, but are in no way limited to, cytokines, growth factors, common beta chain, common gamma chain, and IL-6 cytokine families, adrenomedullin, insulin-like growth factor, epidermal growth factor EGF, fibroblast growth factor FGF, autocrin motility factor, GDF, IGF, PDGF, vascular endothelial growth factor (VEGF (e.g. VEGFA)), growth differentiation factor 9, erythropoietin, activins, TGF-α, TGF-β, bone morphogenetic proteins (BMPs), Hedgehog molecules, Wnt-related molecules, and combinations thereof.

In some aspects, the invention finds use in medical and cosmetic applications for improving the health and appearance of the skin. Thus, the invention finds use in the treatment of degenerative skin conditions due to aging including, but not limited to, loss of skin elasticity (e.g. elastosis, solar elastosis, wrinkles and lines), loss of skin pigmentation (e.g. hypopigmentation, liver spots and lentigos), loss of skin turgor and decreased skin thickness.

The term “skin condition” also refers to disorders of the skin including, but not limited to, degenerative skin condition, skin disease, skin problems, which include, but are not limited, acne, eczema, psoriasis, rosacea, vitiligo, leucoderma, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dennatitis herpetiformis, and Eosinophilic dermatitis. In one specific embodiment, the skin disorder can be mediated by an immunological response. In another specific embodiment, the skin disorder can be a lymphocyte-mediated skin disorder. In another specific embodiment, the skin disorder can be selected from the group of alopecia greata, psoriasis, atopic dermatitis, lupus erythematosis, bullous pemphigoid, and psoriatic plaque. In another specific embodiment, the skin disorder can be psoriasis. In another specific embodiment, the skin disorder can be a chronic skin disorder. In another specific embodiment, the skin disorder can be an autoimmune skin disorder. In another specific embodiment, the skin disorder can be a malignant lymphoid disease that manifests in the skin.

The invention may also be applied in the treatment of injuries to the skin. That is, the invention may be used to improve the cosmetic appearance of, for example, scars, including burns (e.g. chemical and thermal burns). The invention also finds application in the treatment of vascular disorders of the skin such as varicose veins, chronic (long-term) venous insufficiency, thrombophlebitis, and arteriovenous fistula.

As noted above, the invention is practiced by administering a stem cell composition to a patient suffering from a skin condition. As used herein, the terms “administering,” “administered” and “administer” refer to any administration route by which a stem cell composition can be administered to a patient for a therapeutic effect as disclosed herein. For example, the stem cell composition may be administered intravenously, intra-arterially, intramuscularly, intraperitoneally, subcutaneously, intramuscularly, intranasaly, sublingually, or a by combination thereof. In a preferred embodiment, the stem cell composition is administered intravenously. In other aspects of the invention, the stem cell composition may be administered locally. For example, elastosis may be treated by subcutaneously administering a stem cell composition where a cosmetic improvement in the skin is desired.

In a specific, non-limiting example of the invention, a stem cell derivative is administered in the treatment of elastosis. Such exemplary methods may be practiced by obtaining a composition of mesenchymal stem cells from the bone marrow of an allogeneic donor. The stem cells are preferentially expanded and selected as set forth in Example 2, and prepared for administration using a generally accepted pharmaceutical carrier such as PBS. The dosage of stem cells may range between about 1×10⁶ to about 150×10⁶ stem cells. The stem cell composition is then administered intravenously.

In aspects of the invention, the stem cell derivative is preserved. As used herein, the term “preserved” refers to the preservation of a material (e.g. stem cell derivative) by any of the methods disclosed in U.S. Publication No. 2008-0229609, the entire contents of which are incorporated herein by reference. Such processes include, but are not limited to, preservation by evaporation, freeze drying, secondary freeze drying, preservation by foam formation, vaporization, and combinations thereof.

As used herein, the term “vaporization” refers to a movement of molecules into a gas phase by evaporation, sublimation, or boiling.

“Preservation by vaporization” (PBV) is a preservation process that comprises primary drying and stability drying, Primary drying is performed by intensive vaporization (sublimation, boiling and evaporation) of water at temperatures significantly (approximately 10.degree. C. or more) higher than Tg′ from a partially frozen and at the same time overheated (vacuum pressure is below the equilibrium pressure of water vapor) material.

In addition, unlike preservation by foam formation (PFF), preservation by vaporization (PBV) can be very effective for preserving biologicals contained or incorporated within an alginate gel formulation and other gel formulations. A PBV process can be performed by drying frozen gel particles under a vacuum at small negative (on the Celsius scale) temperatures. For such hydrogel systems, vaporization comprises simultaneous sublimation of ice crystals, boiling of water inside unfrozen microinclusions, and evaporation from the gel surface.

It should be noted that preserved stem cell derivatives include such derivatives in dry (e.g. containing less than 10% water by weight) and reconstituted form. For example, preserved stem cell derivatives include vaporized or lyophilized stem cell derivatives which have been reconstituted in an solution (e.g. pharmaceutically acceptable carrier) for administration to a patient.

In an exemplary, non-limiting embodiment of the invention, a skin disorder in a patient, such as elastiosis, is treated by topically administering to the patient a vaporized stem cell derivative that comprises a therapeutic amount of at least one stem cell factor, including VEGF. In such an embodiment, the stem cell derivative may be obtained from medium that has been conditioned by the growth of mesenchymal stem cells under low oxygen conditions. Such conditioned medium may be collected, concentrated, and then preserved by vaporization. The avaporized preserved conditioned medium, containing VEGF, may then be stored for a prolonged period of time before being reconstituted (using a gel for example) for administration in the treatment or rejuvenation of the skin.

### Example 1

### Seeding Human Bone Marrow MSC for Cytokine Accumulation

1a. Collection of Sample

Human bone marrow was collected from consented donor. Plasma, extraneous material (bone fragments, fat), and erythrocytes were removed from the BMS aspirate. The obtained suspension of nucleated cells was plated in plastic dishes in growth medium DMEM/F12 (1/1) (Gibco, Grand Island), containing 20% fetal calf serum (HyClone, USA), 2 mM glutamine, and antibiotics. The plating density of the primary cell suspension was 500,000-1,000,000 cells/cm² on average. Cells were cultured under standard conditions (at 37° C. in an atmosphere of 5% CO₂). After a day, unattached cells were removed, and attached cells were incubated to 70-80% confluence, which generally takes from 10 to 20 days. The culture medium was replaced every 3 days.

1b. Cell Culture

The mononuclear cells were isolated from fresh specimen using Histopague and seeded into Petri dishes. The cells were expanded in culture medium (DMEM-F12+15 mM HEPES+15% FBS+FGF2+40 mg/L heparin+ITS+GlutaMax). The cells were tested for human pathogens and were divided into two lots at passage 2: (a) cells further under under 5% oxygen conditions, and (b) cells grown under 21% oxygen.

1c. Cytokine Accumulation Medium

MSC were transferred to the a cytokine accumulation medium (DMEM-F12+15 mM HEPES+40 mg/L heparin+GlutaMax+5%) and incubated for an additional three days before the collection of the conditioned medium. Some MSC samples were stimulated with an LPS solution in the 10-1,000 ng/ml range (200 ng/ml LPS being preferred a more popular concentration). Medium was refreshed once 62 hours prior to the collection of the cell free medium. 480 ml of medium was collected and spun for 20 minutes at 4,000 rpm. The liquid fraction was filtered through a 200 nm filter. Medium was then concentrated 4 times using a 5 kDa PES membrane (Millipore stirred cell, 300-400 rpm, 40-45 psi, pressurized gas=1% O₂+7% CO₂+92% N₂). Concentrate medium was filtered through a 200 nm membrane.

1d. Sample Preservation

Freeze-drying: 10 grams of conditioned medium from above [1×MSC-CM+200 μg/ml heparin] were mixed with 10 gram of sterile buffer for freeze-drying→10 g=˜9.7 ml. The mixture was pipetted into 20 vials, 1 ml per vial and placed into lyophilizing chamber. The drying protocol was recorded in the computer (pressure vs. temperature vs. time).

Evaporative-drying: 10 grams of conditioned medium [1×MSC-CM+200 μg/ml heparin] were mixed with 10 gram of sterile buffer for evaporative drying→10 g=˜8 ml. The mixture was pipetted into 20 vials, 1 ml per vial and placed into vacuum chamber. The drying protocol was recorded in the computer (pressure vs. temperature vs. time).

Frozen control for evaporative-drying: 10 grams of [1×MSC-CM+200 μg/ml heparin] were mixed with 10 grams of sterile buffer for evaporative drying. The mixture was pipetted into ˜20 vials, 1 ml-1.5 ml per vial and placed into a −80° C. freezer.

All preserved, concentrated supernatants (dried, freeze-dried and liquid) were kept for 10 days at +4° C. On Monday, Mar. 29, 2010, one-half of the vials were transferred into +37° C. incubator.

### Example 2

### Preservation of Human VEGF from MSC-Conditioned Medium

Human bone marrow mesenchymal cells (BM-MSC) were purchased from Lonza and seeded into two triple-layer flasks (250,000 cells/500 cm²/125 ml/flask→500 cells/cm²) and cultured for 7 days in 21% oxygen without changing the medium (DMEM-F12+15 mM HEPES+15% FBS+FGF2+40 mg/L heparin+ITS+GlutaMax). Conditioned medium was collected (˜250 ml).

Cells were removed from the flasks using Tryp-LE protease. 27,000,000 cells were harvested from 2 flasks. Cells were divided into 2 equal parts and grown for five passages under 21% oxygen in DMEM-F12+15 mM HEPES+15% FBS+FGF2+40 mg/L heparin+ITS+GlutaMax.

250 ml of the conditioned medium (MSC-CM) was centrifuged to remove cell debris, and then passed through sterile filter (200 nm pore size). MSC-CM (80 ml) was concentrated using Pierce membrane cones (9 kDa, 20 ml, Pierce P/N PI89885A, 25/pack, $250). The reason for using 9 kDa membrane cones was to increase VEGF concentration and improve the pH80 ml of MSC-CM was distributed into 4 cones, and centrifuged at 4,000 rpm for 40 minutes. At that speed, filtration rate was 5 ml in 10 minutes. (NB. 4,000 rpm is the maximum speed allowed for the rotor). The centrifuge does not have refrigeration. as a result, the tubes were warm to the touch after 40 minutes of spinning (+35° C. to +40° C.). 80 ml of MSC-CM were reduced to 7 ml (˜10× volume reduction). 7 ml of 10×concentrate were diluted 1:1 with PBS. Tris, pH 7.8 to improve pH (100 mM Tris final concentration.

Conditioned medium was divided into 2 parts: Part #1: undiluted MSC-CM (1×, black marker labels). Part #2: MSC-CM concentrated 5-fold (5×, red marker labels). Portions of the 5×MSC-CM were then treated as follows: CM-5×[+3° C.] were preserved as a dried foam and then stored for 9 days at 3° C.; CM-5×[+37° C.] cells were preserved as a dried foam and then stored for 9 days at 37° C.; and CM-5×[−80° C.] which were stored in liquid form at −80° C.

The 5×MSC-CM was preserved as a dried foam according to the materials and methods disclosed in U.S. Publication No. 2008-0229609, the contents of which are incorporated herein by reference.

### Analysis of MSC-CM

After storage as set forth above, the preserved samples were then subjected to standard ELISA analysis. The following results were obtained:

Quadratic curve fit-->R̂2=1.000

| | | Out- | | dilu- | VEGF | VEGF | Dilution |
|---|---|---|---|---|---|---|---|
| # | CV % | liers | Description | tion | (diluted) | (neat) | linearity |
| 01 | 1.7 | | CM-5 × | 4 | 466 | 1,864 | 1 |
| | | | [+3° C.] | | | | |
| 02 | 15.5 | | CM-5 × | 8 | 441 | 3,528 | 1.9 |
| | | | [+3° C.] | | | | |
| 03 | 0.3 | | CM-5 × | 16 | 332 | 5,312 | 2.8 |
| | | | [+3° C.] | | | | |
| 04 | 11.0 | | CM-5 × | 32 | 191 | 6,112 | 3.3 |
| | | | [+3° C.] | | | | |
| 05 | 10.2 | | CM-5 × | 4 | 459 | 1,836 | 1 |
| | | | [+37° C.] | | | | |
| 06 | 16.6 | | CM-5 × | 8 | 440 | 3,520 | 1.9 |
| | | | [+37° C.] | | | | |
| 07 | 0.9 | | CM-5 × | 16 | 311 | 4,976 | 2.7 |
| | | | [+37° C.] | | | | |
| 08 | 9.6 | | CM-5 × | 32 | 184 | 5,888 | 3.2 |
| | | | [+37° C.] | | | | |
| 09 | 6.2 | | CM-5 × | 4 | 486 | 1,944 | 1 |
| | | | [−80° C.] | | | | |
| 10 | 13.9 | | CM-5 × | 8 | 501 | 4,008 | 2.1 |
| | | | [−80° C.] | | | | |
| 11 | 0.3 | | CM-5 × | 16 | 336 | 5,376 | 2.8 |
| | | | [−80° C.] | | | | |
| 12 | 2.8 | | CM-5 × | 32 | 210 | 6,720 | 3.5 |
| | | | [−80° C.] | | | | |

### Example 3

### Preservation of Human MSC

BM-MSC that were removed from the flasks and separated from Example 2 were separated into two groups. Group 1 cells were suspended in 10 ml of 20% DMSO in Hanks' BSS, incubated on ice for 30 minutes to increase intracellular concentration of DMSO, and transferred into −80° C. for storage. Group 2 cells were pelleted at 300×G, and resuspended in approximately 0.5 ml of preservation medium #1 (3-oxy-methyl-D-glucose+gelatin) and dried in vacuum according to the method disclosed in U.S. Publication No. 2008-0229609, the disclosure of which is incorporated herein by reference.

I claim:

1. A method for treating a skin disorder in a patient comprising:
a. providing a vaporized stem cell derivative; and
b. administering the vaporized stem cell derivative to the patient;
c. wherein administering the vaporized stem cell derivative treats the patient's skin disorder.

2. The method of claim 1, wherein the vaporized stem cell derivative comprises stem cells, stern cell factors, microvesicles, or a combination thereof.

3. The method of claim 2, wherein the vaporized stem cell derivative is administered intravenously, intra-arterially, intramuscularly, intraperitoneally, subcutaneously, intramuscularly, topically, or a combination thereof.

4. The method of claim 3, wherein the stem cell derivative is obtained from mesenchymal stem cells.

5. The method of claim 4, wherein the mesenchymal stem cells are autologous, allogeneic, or a combination thereof with respect to the patient.

6. The method of claim 5, wherein the mesenchymal stem cells are derived from human bone marrow or adipose.

7. The method of claim 6, wherein the skin disorder is selected from elastosis, hypopigmentation, liver spots, hypopigmentation, and combinations thereof.

8. The method of claim 6, wherein the mesenchymal stem cells are grown under low serum, low oxygen conditions.

9. The method of claim 8, wherein the mesenchymal stem cells are upregulated for the expression of VEGF.

10. The method of claim 9, wherein the stem cell derivative comprises at least one stem cell factor.
